(19) United States
(12) Patent Application Publication
(10) Pub. No.: US 2008/0206365 A1
(43) Pub. Date: Aug. 28, 2008

(54) Diterpene compounds having an atisane framework, compositions thereof, and methods of production

(76) Inventors: Yoshihisa Tachibana, Tokyo (JP); Shoichiro Kurata, Aoba-ku (JP); Yoshiteru Oshima, Taihaku-ku (JP); Kazunori Ueda, Aoba-ku (JP); Haruhisa Kikuchil, Taihaku-ku (JP)

Correspondence Address: CLIFFORD CHANCE US LLP, 31 WEST 52ND STREET, NEW YORK, NY, 10019-6131, US

(21) Appl. No.: 11/680,498

(22) Filed: Feb. 28, 2007

(51) Int. Cl. A61K 38/00; A61K 31/075; A61P 43/00; A61K 31/21

(52) U.S. Cl. 424/725; 514/511; 514/715

(57) ABSTRACT

Provided are a certain compounds and compositions useful for inhibiting or activating the production of various types of cytokines. Also provided are certain compounds and compositions for preventing or treating various diseases attributable to abnormal cytokine production or compromised immunity.

Provided are certain diterpene compounds having an artisane framework, as well as compositions comprising certain diterpene compounds having an artisane framework, methods of producing those compounds and compositions, and methods for their use.

Cytokines are low molecular weight proteins secreted from cells that act in intercellular signal transduction. The physiological functions of cytokines include control of immune responses, anticarcinogenic effects, anti-viral effects, and regulation of cell growth-differentiation. Various diseases are known to be contracted or to develop when the balance of production is lost. For example, chronic diseases such as rheumatism, osteoporosis, arteriosclerosis, and diabetes complications develop when cytokines are overproduced. Furthermore, cytokines act as immunotherapeutic agents and as hematopoietics. Cytokine administration provides therapeutic effects against various diseases. However, isolation of large volumes of cytokines from tissue as well as production of cytokines at high-purity has been difficult, and cytokine administration from external sources has not been practical. For that reason, a cytokine production control agent that can modulate cytokine production in vivo is desired.

The screening method using Drosophilia is known to be a sensitive method of screening the effective constituents (e.g. cytokines) acting on the human innate immune system. Compounds that block the production of chemokine, one type of cytokine, have been discovered using this method. However, a large number of cytokines exist in addition to chemokine, and there are cases in which the activation or inhibition of cytokine production is required, depending on the application. Accordingly, the production of cytokines in vivo may not be adequately controlled by relying solely on the compound discussed in Gazette of Japanese Kokai Publication Hei-2005-187451. Thus, it would be desirable to have cytokine production control agents capable of modulating the production of various cytokines.

Provided is a pharmaceutical composition comprising at least one diterpene compound of formula (1)

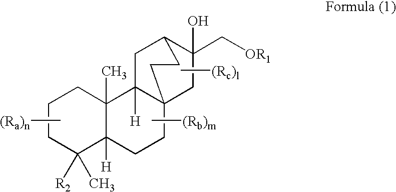

wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

Also provided is a method for treating or preventing a disease attributable to abnormal production of at least one cytokine comprising administering to a subject in need thereof a therapeutically effective amount of at least one pharmaceutical composition described herein.

Also provided is a composition for controlling cytokine production comprising at least one diterpene compound of formula (1)

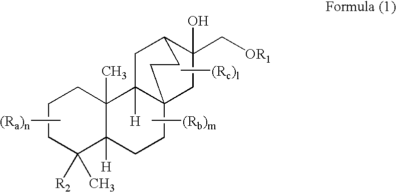

wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

Also provided is food comprising a composition for regulating cytokine production described herein.

Also provided is a method of producing one or more diterpene compounds of formula (1) comprising extracting parts of Annona Cherimola Mill., and isolating from the resulting extract one or more diterpene compounds of formula (1)

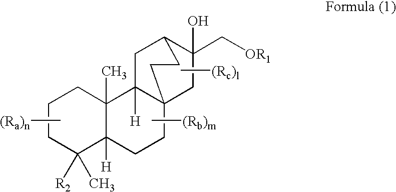

wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

Also provided is a diterpene compound of formula (3)

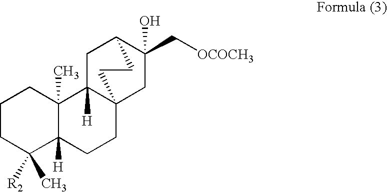

wherein
R₂ is selected from optionally substituted C₁ to C₆ alkoxycarbonyl, optionally substituted
C₁ to C₆ acyl, and optionally substituted C₁ to C₆ hydrocarbon.

### BRIEF DESCRIPTION OF THE DRAWINGS

FIG. 1 is a diagram showing the isolation procedures of diterpene compounds from Annona Cherimola Mill. extract.

FIG. 2 is the specific rotation, ¹H-NMR, ¹³C-NMR, LREIMS and HREIMS data of AC-4.

FIG. 3 is the specific rotation, ¹H-NMR, ¹³C-NMR, LREIMS and HREIMS data of AC-5.

FIG. 4 is the specific rotation, ¹H-NMR, ¹³C-NMR, LREIMS and HREIMS data of AC-6.

FIG. 5 is a graph showing the IL-8 production control effects of diterpene compounds 1 to 6.

FIG. 6 is a graph showing the MCP-1 production control effects of diterpene compounds 1 to 6.

FIG. 7 is a graph showing the spontaneous immunoactivity, cytotoxicity, and transcription-translation activity of diterpene compounds 1 to 6.

As used in the present specification, the following words and phrases are generally intended to have the meanings as set forth below, except to the extent that the context in which they are used indicates otherwise.

“C₁ to C₆ hydrocarbon” refers to saturated or unsaturated straight-chain or branched-chain hydrocarbon having 1 to 6 carbon atoms. Examples include alkyls such as methyl, ethyl, n-propyl, iso-propyl, n-butyl, iso-butyl, sec-butyl, tert-butyl, n-pentyl, 1,1-dimethylpropyl, 1,2-dimethylpropyl, 2,2-dimethylpropyl, 1-ethylpropyl, n-hexyl, 1-ethyl-2-methylpropyl, 1,1,2-trimethylpropyl, 1-ethylbutyl, 1-methylbutyl, 2-methylbutyl, 1,1-dimethylbutyl, 1,2-dimethylbutyl, 2,2-dimethylbutyl, 1,3-dimethylbutyl, 2,3-dimethylbutyl, 2-ethylbutyl, 2-methylpentyl, 3-methylpentyl; alkenyls such as vinyl, allyl, 1-propenyl, isopropenyl, 2-methyl-1-propenyl, 1-butenyl; and alkynyls such as ethynyl, 1-propynyl, 2-propynyl, 1-butynyl, 2-butynyl, 3-butynyl, 1-ethynyl-2-propynyl, and 1-methyl-2-propynyl. In certain embodiments, “C₁ to C₆ hydrocarbon” refers to methyl, ethyl, n-propyl, iso-propyl, n-butyl, iso-butyl, sec-butyl, tert-butyl, and n-pentyl.

“C₁ to C₆ acyl” refers to groups in which carbonyls are bound to hydrogen or a straight-chain or branched-chain hydrocarbons having 1 to 5 carbon atoms. Examples include aldehyde (formyl), acetyl, propionyl, butyryl, iso-butyryl, valeryl, iso-valeryl, pivaloyl, and the like. In certain embodiments, “C₁ to C₆ acyl” refers to aldehyde, acetyl, propionyl, butyryl, iso-butyryl, valeryl, iso-valeryl, and pivaloyl.

“C₁ to C₆ alkoxycarbonyl” refers to a group having a carbonyl bound to a C₁ to C₅ alkoxy. Here, C₁ to C₅ alkoxy connotes groups in which oxygen atoms are bound to a straight-chain or branched-chain hydrocarbons having 1 to 5 carbon atoms. Examples of C₁ to C₅ alkoxy include methoxy, ethoxy, n-propoxy, iso-propoxy, n-butoxy, iso-butoxy, sec-butoxy, tert-butoxy, n-pentyloxy, iso-pentyloxy, sec-pentyloxy, neopentyloxy, 1-methylbutoxy, 2-methylbutoxy, 1,1-dimethylpropoxy, 1,2-dimethylpropoxy, n-hexyloxy, iso-hexyloxy, 1-methylpentyloxy, 2-methylpentyloxy, 3-methylpentyloxy, 1,1-dimethylbutoxy, 1,2-dimethylbutoxy, 2,2-dimethylbutoxy, 1,3-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy. Examples of “C₁ to C₆ alkoxycarbonyls” include methoxycarbonyl, ethoxycarbonyl, n-propoxycarbonyl, and iso-propoxycarbonyl.

By “optional” or “optionally” is meant that the subsequently described event or circumstance may or may not occur, and that the description includes instances where the event or circumstance occurs and instances in which it does not. For example, “optionally substituted C₁ to C₆ hydrocarbon” encompasses both “C₁ to C₆ hydrocarbon” and “substituted C₁ to C₆ hydrocarbon”.

“Substituted” means that one or more hydrogen is displaced by another substituent. Examples of such substituents include hydroxyl, amino, halogens (for example, fluorine, chlorine, bromine, iodine), cyano, nitro, carboxyl, oxo, and alkoxy.

The term “therapeutically effective amount” means an amount effective, when administered to a human or non-human patient, to provide a therapeutic benefit such as amelioration of symptoms, slowing of disease progression, or prevention of disease e.g., a therapeutically effective amount may be an amount sufficient to decrease the symptoms of a disease.

“Patient” refers to an animal, such as a mammal, that has been or will be the object of treatment, observation or experiment. The methods of the invention can be useful in both human therapy and veterinary applications. In some embodiments, the patient is a mammal; in some embodiments the patient is human; and in some embodiments the patient is a non-human animal.

Provided are compositions comprising compounds of Formula (1):

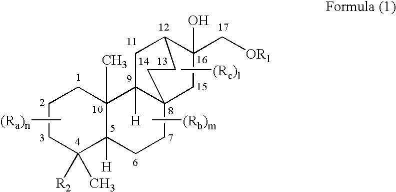

wherein

R₁ is selected from hydrogen, optionally substituted C₁ to C₆ acyl, and optionally substituted C₁ to C₆ hydrocarbon;

R₂ is selected from optionally substituted C₁ to C₆ alkoxycarbonyl, optionally substituted C₁ to C₆ acyl, and optionally substituted C₁ to C₆ hydrocarbon;

Rₐ, R_{b}, and R_{c} are independently optionally substituted C₁ to C₆ hydrocarbon;

n is an integer selected from 0, 1, 2, and 3;

m is an integer selected from 0, 1, and 2; and

l is an integer selected from 0, 1, 2, 3, 4, and 5.

Rₐ, R_{b}, and R_{c} in formula (1) are substituents that may displace a hydrogen bound to carbon at positions 1 to 3, position 6, position 7, or positions 11 to 15. In certain embodiments, n is 0. In certain embodiments, m is 0. In certain embodiments, l is 0. In certain embodiments, m, n, and l are 0.

In some embodiments, R₁ is hydrogen or acetyl. In some embodiments, R₁ is acetyl. In some embodiments, R₁ is hydrogen.

In some embodiments, R₂ is selected from methoxycarbonyl, aldehyde, and hydroxymethyl.

In some embodiments, R₂ is methoxycarbonyl.

In some embodiments, R₂ is aldehyde or hydroxymethyl.

In some embodiments, R₂ is aldehyde.

In some embodiments, R₁ is hydrogen and R₂ is aldehyde.

In certain embodiments, the diterpene compounds represented by formula (1) have the structure represented by formula (2),

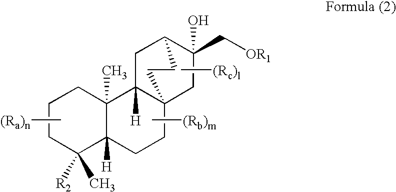

wherein R₁, R₂, Rₐ, R_{b}, R_{c}, n, m, l are as described for compounds of Formula (1).

Also provided are certain diterpene compounds represented by Formula )(3.

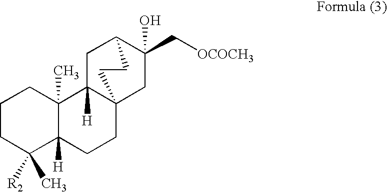

wherein R₂ is as described for compounds of Formula (1).

In certain embodiments, the compound of Formula (I) is chosen from

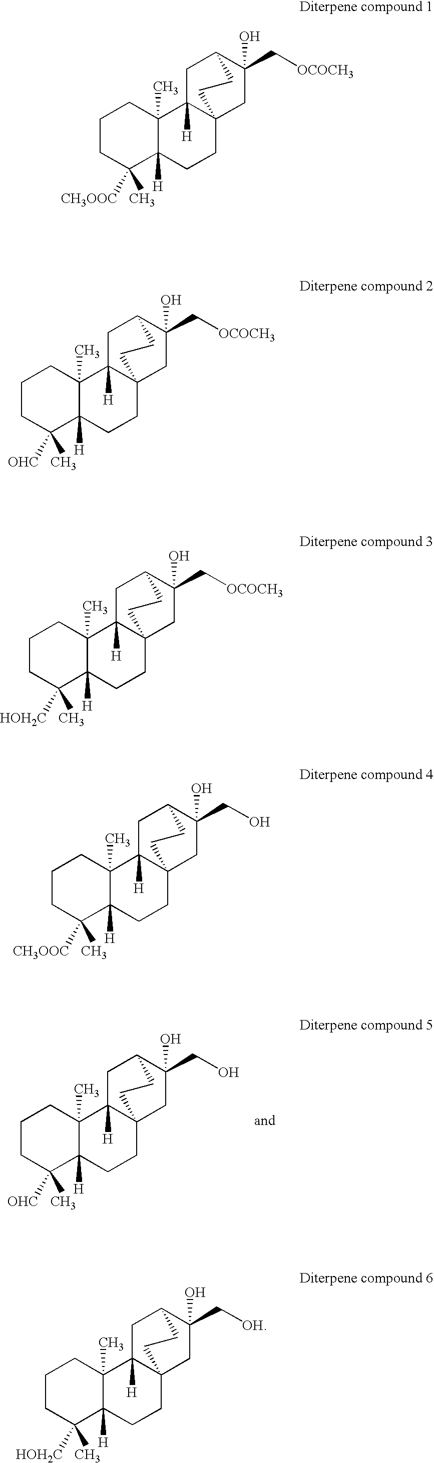

The diterpene compounds represented by formula (1) exhibit control (inhibition or activation) of cytokine production. In some embodiments, the compounds described herein exhibit control of cytokines whose expression is induced by transcription factors such as NF-κB or NF-AT. Examples of such cytokines include IL-1 to IL-23, IFN-α, IFN-β, IFN-γ, TNF-α, TNF-β, MCP-1, MCAF, RANTES, MIP-1, SCF, GM-CSF, G-CSF, M-CSF, TGF-β, PDGF, EGF, LIF, and GRO-α. In some embodiments, other cytokines are controlled.

The diterpene compounds represented by formula (1) can regulate the cytokine production control effect through the suitable selection of R₁ and R₂. For example, the cytokine production control effect of the diterpene compounds represented by formula (1) increases when R₁ is hydrogen or acetyl and more particularly, when R₁ is acetyl. For example, the diterpene compounds represented by formula (1) activate cytokine production at low concentration ranges when R₂ is methoxycarbonyl. Conversely, the diterpene compounds represented by formula (1) inhibit cytokine production when R₂ is aldehyde or hydroxymethyl. Diterpene compound 5 (discussed below) in which R₁ is hydrogen and R₂ is aldehyde loses cytokine production control activity or converts cytokine production control activity at high concentration regions. In this manner, diterpene compounds represented by formula (1) can exhibit various cytokine production control effects through suitable combinations of R₁ and R₂.

The diterpene compounds represented by formula (1) exhibit inhibition or activation of cytokine production and may be used as the active ingredients of pharmaceutical compositions for treating or preventing diseases attributable to abnormal cytokine production in humans and animals other than humans. Examples of diseases attributable to such abnormal cytokine production include viral infections, myocardial infarction, rheumatism, osteoporosis, arteriosclerosis, diabetes complications, sepsis, multiple myeloma, cervical cancer, post-organ transplant rejection response, hepatocirrhosis, acquired immune deficiency syndrome (AIDS), multiple sclerosis (MS) and the like.

When the diterpene compounds represented by formula (1) activate cytokine production, in vivo immunity can be activated by their administration. Consequently, they can be used as the active ingredients of pharmaceutical compositions for the prevention of diseases attributable to compromised immunity. Influenza and Spanish flu are examples of diseases attributable to such compromised immunity. In addition, infections can be inhibited by administering the diterpene compounds represented by formula (1) to patients whose resistance is compromised by acquired immunity inhibitors (for example, tacrolimus, cyclosporine, etc.) that are used following organ transplantation.

Since the diterpene compounds represented by formula (1) inhibit or activate cytokine production, they can be used as compositions for regulating or controlling cytokine production (cytokine production inhibitors or cytokine production activators). Cytokine production controllers containing the diterpene compounds represented by formula (1) can be added to health foods (including beverages) or feed as, for example, immunopotentiators. In addition, cytokine production controllers containing the diterpene compounds represented by formula (1) can be added to agrochemicals as insecticides. When crop diseases develop, insects whose immune function is compromised become infected and die without spreading germs and the like to healthy crops since the immune function of insects that collect on crops can be compromised by spraying the cytokine production controllers on crops, and this permits the onset of damage to be minimized. In addition, insects that pathogenically damage crops but whose immune function is compromised can be easily eradicated by using a smaller amount of insecticide than usual.

When using the diterpene compounds represented by formula (1) in pharmaceutical compositions or composition for regulating cytokine production, the diterpene compounds represented by formula (1) can be administered to humans or animals other than humans as is or they can be administered with common carriers or additives. The dosage can be suitably determined as a function of the diterpene compound, the objective of administration, and the target of administration (patient symptoms, age, weight, sex, etc.). For example, a dosage range of 1 mg to 2 g per day to an adult, such as a range of 50 mg to 1000 mg, may be used. In this case, administration may be once daily or divided into numerous sessions daily.

In certain embodiments, the cytokine production control effect of diterpene compounds represented by formula (1) may vary depending upon the concentration of compound added. For example, diterpene compound 5 can regulate the cytokine production control effect through suitable adjustment of the dosage.

Both oral and non-oral administration is possible. Furthermore, the dosage form can be suitably selected as a function of the administration method, administration objective, and administration subjects (patient symptoms, age, weight). Permissible examples include tablets, capsules, granules, powder, troches, ointments, creams, emulsions, suspensions, suppositories, or injections. These pharmaceutical compositions can be produced by common drug-production technology (for example, methods established by the Pharmacopoeia of Japan). These pharmaceutical compositions may contain pharmaceutically acceptable additives. Examples of such pharmaceutically acceptable additives include excipients, binders, lubricants, disintegrating agents, dissolution promoters, suspending agents, and emulsifiers.

The diterpene compounds represented by formula (1) can be produced by a method comprising the steps of extracting parts of Annona Cherimola Mill., a plant of the squamosa family, squamosa genus, and isolating one or more diterpene compounds represented by formula (1) from the resulting extract. The Annona Cherimola Mill. used as starting material may be fresh or dried, but finely cut dried material is used in certain embodiments. Any extraction site of Annona Cherimola Mill. can be used so long as it is a site with a sufficient diterpene compound content. Examples of extraction sites include the fruit, the stalk, leaves, root, flowers, seeds, or mixtures thereof, but in certain embodiments, fruit is used as the extraction site, and in certain embodiments, the pulp excluding the skin and seeds is used.

Any method of extraction from Annona Cherimola Mill. can be used, such as liquid-phase extraction and gaseous-phase extraction. Any extraction solvent can be used in liquid-phase extraction so long as it permits extraction of the diterpene compounds represented by formula (1) that are contained in Annona Cherimola Mill. However, in certain embodiments, an organic solvent is used as the extraction solvent because many of the diterpene compounds represented by formula (1) are insoluble in water. In certain embodiments, lower alcohols such as ethanol and methanol are used. The extraction temperature and extraction duration are suitably selected as a function of the extraction site of Annona Cherimola Mill., the degree of grinding/cutting, the extraction method, and the extraction solvent. In certain embodiments, an extraction temperature range of 4 to 60° C. is used. In certain embodiments, the extraction duration is about 30 minutes to about 10 days.

Any method of isolating the diterpene compounds from the resulting extract can be used, such as separation via column chromatography, including high-performance liquid chromatography and silica-gel column chromatography. Permissible development solvents used in column chromatography are conventional development solvents including n-hexane, benzene, ethyl acetate, ethanol, methanol, acetone, ether, and chloroform. The fractions containing the target compounds are selected from among the plurality of fractions separated by column chromatography when isolating compounds using column chromatography. The fractions containing the one or more diterpene compounds represented by formula (1) can be selected by measuring the cytokine production inhibition or activation in each fraction followed by selecting the fractions that exhibit the strongest cytokine production inhibition or activation. Cytokine production inhibition or activation in each fraction of the chromatographic separation may be evaluated by utilization of a spontaneous immunoactivity evaluation system that employs Drosophilia as discussed in Gazette of Japanese Kokai Publication Hei 2004-121155.

Following the step of isolating the one or more diterpene compounds represented by formula (1) from an extract of Annona Cherimola Mill., a step of converting one or more of the functional groups, for example, substituents bound at position 4 and position 17, of the isolated one or more diterpene compounds into other substituents (R₁, R₂) by known methods may be incorporated. The compound, through functional groups conversion, may be converted from the diterpene compounds extracted from Annona Cherimola Mill. into diterpene compounds having a desired cytokine production control action. Known techniques of conversion include reduction processing and deactylation.

The present invention is explained below through examples, but the present invention is not restricted to these examples. The equipment, reagents, notation methods used in the examples are presented below.

The specific rotation was measured using a model P-1030 polarimeter from JASCO Corporation. The mass spectrum was measured using a model JMS-DX303 mass spectrometer and model JMS-700 mass spectrometer from JEOL Ltd. NMR spectra were measured using a model ECA-600 and a model AL-400 nuclear magnetic resonance device from JEOL Ltd, and TMS was used as the internal standard. The chemical shift values are represented in ppm. Multiplicities are represented as follows: singlet: s, doublet: d, triplet: t, quartet: q, double doublet: dd, multiplet: m, broad signal: br. Column chromatography used Silica gel 60 (70-230 mesh ASTM, Merck), Cosmosil 140C₁₈-OPN (Nacalai). GPC HPLC used LC-908W (Japan Analytical Industry Co., Ltd.). The column used JAIGEL-GS310 (φ20 mm×600 mm) (Japan Analytical Industry Co., Ltd.). JAIGEL-IH (φ21.5 mm×500 mm) (Japan Analytical Industry Co., Ltd.). TLC used TLC aluminum sheets Silica gel 60F₂₅₄ (0.25 mm, Merck), TLC aluminum sheets RP-18 F₂₅₄ₛ (0.25 mm, Merck). Detection was carried out by hot color development following spraying with an anisaldehyde sulfuric acid solution and UV (254 and 365 nm) irradiation fluorescence. The reagents were commercial reagents that were used as is.

### EXAMPLES

### Example 1

### Evaluation of Spontaneous Immunoactivity

The following was utilized in selecting the fractions containing the diterpene compounds represented by formula (1) from among a plurality of fractions separated by column chromatography in the following examples. The amount of expression of β-galactosidase, a reporter protein of the diptericin gene that is an antibacterial peptide of the spontaneous immune system of each sample, was measured to evaluate the spontaneous immunoactivity of each sample (fraction). Specific procedures are presented below.

### (i) Preparation of Culture Medium

A culture medium comprising Schneider's Drosophilia culture medium (GIBCO) (culture medium for culturing Drosophilia cells) to which 20% calf serum (Valley Biomedical Inc.) and 1% antibiotic (Antibiotics-Antimycotic: GIBCO) were added was prepared as the culture medium for culturing Drosophilia fat bodies. The lipopolysaccharide (LPS) used in activate spontaneous immunity was first dissolved in purified water to reach a concentration of 1 mg/mL, followed by addition to the culture medium so that the final concentration would reach 1% (10 μg/ml). In addition, the sample was converted into a solution using dimethyl sulfoxide (DMSO) that was then added so that the final concentration would reach 0.5% (0.5 μg/mL).

### (ii) Necropsy and Incubation

Only female Drosophilia were selected. They were decapitated and the fat bodies, which are the organs that produce antibacterial peptide, were exposed. The resulting fat bodies were added, one each, to individual wells of a 96-well plate containing 100 μL of culture medium per well, followed by incubation for 12 hours at 25° C. Six Drosophilia were used per sample in incubation.

(iii) Preparation of Analysis Sample used in β-galactosidase Quantification

Following incubation for 12 hours, the fat bodies were added to a 500 μL Eppendorf tube containing 200 μL of reaction buffer (60 mM Na₂HPO₄, 40 mM NaH₂PO₄, 10 mM KCl, 1 mM MgCl₂, pH 7.8) and homogenized using an ultrasonic homogenizer (ULTRASONIC PROCESSOR XL: MISONIX) to complete β-galactosidase extraction. The extract was subjected to centrifugal separation (9170×g, 10 min., 4° C. MX-300: TOMY). The supernatant was collected (160 μL) and was used as the analysis sample.

(iv) β-galactosidase Quantification

The β-galactosidase in the analysis sample obtained in (iii) was quantified by the enzyme reaction. Solutions in which the β-galactosidase concentration interval was 100 ng/mL, 10 ng/mL, 1 ng/mL, 100 μg/mL, and 10 μg/mL were prepared by diluting with β-galactosidase with 0.1% BSA (bovine serum albumin)+reaction buffer as the standard solution for generating a calibration curve. Into each measurement tube was injected 20 μL of standard solution and of analysis sample, followed by the addition thereto of 80 μL of Galacton plus (TROPIX), an enzyme substrate, that had been diluted 80-fold with reaction buffer. It was then stirred at room temperature for precisely one hour. Subsequently, 100 μL of Emerald II (TROPIX), an enhancer, that had been diluted 80 fold with 0.25 M NaOH was added to the standard solution and the analysis solution. This was immediately used to measure the chemical light emission of the standard solution and the analysis solution via luminometer (Microplate Luminometer LB96V: Belthold). The β-galactosidase content in the analysis sample was quantified using the calibration curve that had been generated using the standard solution.

### (v) Quantification of Protein by Bradford Method

The protein levels in each analysis sample were quantified to compensate for individual differences in fat body size among Drosophilia, and the β-galactosidase level per unit protein was computed. Solutions in which the BSA concentration was adjusted to 0.5, 0.4, 0.25, 0.125, and 0.05 (mg/mL) by diluting 1 mg/mL of BSA with purified water were prepared as standard solutions for generating calibration curves. The light absorption coefficients of the analysis sample and of the standard solution at 595 nm were measured using a MICRO PLATE READER MODEL680 (BIO-RAD) following injection of 10 μL each of standard solution and of analysis sample into 96 well plates, the addition of 200 μL of 5-fold diluted dye reagent (BIO-RAD) and storage for approximately 10 minutes at room temperature. The protein level in the analysis sample was quantified using the calibration curve that was generated using the standard solution, and the β-galactosidase level per unit protein in the analysis sample was computed.

### (vi) Evaluation

Six Drosophilia were used per sample in incubation. Among the analysis samples, those with the most and least β-galactosidase production per unit protein were excluded. The mean level of β-galactosidase production per unit protein of four analysis samples remaining was computed and that was used in evaluating the spontaneous immunoactivity. The mean β-galactosidase production level per unit protein of control sample comprising a culture medium to which only DMSO and LPS were added was taken as 100%, and the mean β-galactosidase production level per unit protein of blank sample comprising culture medium to which only DMSO had been added was taken as 0%. The β-galactosidase production level per unit protein of each sample was expressed as a relative value (%) relative to the control sample, and this served as the value for evaluating the spontaneous immunoactivity of each sample.

### Example 2

### Evaluation of Cytotoxicity (Measurement of Cell Survival Rate)

The cytotoxicity of the samples was evaluated in the following examples.

### (i) Culture Medium Preparation

A culture medium comprising Schneider's Drosophilia culture medium (GIBCO) to which 20% (V/v) calf serum (Valley Biomedical Inc.) was added so that Antibiotics-Antimycotic (GIBCO) would reach 1% (v/v) was prepared. Sample dissolved in DMSO was added to this culture medium so that the final concentration would reach 0.5% (v/v). In addition, a control sample was prepared in which DMSO alone was added to culture medium.

### (ii) Cell Incubation

Into each well of a 96-well plate was injected 100 μL of culture medium to which sample had been added. S2 cells were sown therein so as to reach 2×10⁵ cells/well. In addition, a blank sample was prepared in which cells were not sown. That was followed by incubation for 24 hours at 25° C. One sample of cells was incubated in six wells.

(iii) Measurement of Number of Live Cells

After incubation for 24 hours, 10 μL of MTT sample (sample for measuring the number of live cells SF: nacalai tesque) was injected in each well. Injection was followed by immediately by measurement of the light absorption coefficient at 450 nm using a MICRO PLATER READER MODEL 680 (BIO-RAD) (taken as the light absorption coefficient at 0 h). That was followed by incubation for 4 hours at 25° C. and remeasurement of the light absorption coefficient at 450 nm (taken as the light absorption coefficient as 4 h).

### (iv) Evaluation

The light absorption coefficient at 0 h was subtracted from the light absorption coefficient at 4 h for each well, and the change in the light absorption coefficient of control samples comprising culture medium to which only DMSO had been added was taken as 100% while the change in the light absorption coefficient of blank samples comprising culture medium alone in which cells had not been sown was taken as 0%. The change in the light absorption coefficient of each well was represented as a relative figure (%) versus the control sample. The mean survival rate of live cells in six wells was computed and this was taken as the survival rate of live cells in the sample.

### Example 3

### Production and Identification of Diterpene Compound 1 Represented by Formula (1)

### (i) Production of Diterpene Compound 1: Extraction Step

To 386.61 g of freeze-dried Annona Cherimola Mill. pulp was added 2 liters of methanol (MeOH), followed by extraction for one day at room temperature while stirring at 70 rpm. The extracted MeOH was filtered off and concentrated under vacuum to yield 169.51 g of MeOH extract. To this MeOH extract was added 1300 mL of water, followed by extraction three times using 1300 mL of ethyl acetate (EtOAc). The EtOAc layer was removed under vacuum to yield 21.5 g of EtOAc soluble fraction.

### (ii) Production of Diterpene Compound 1: Isolation Step

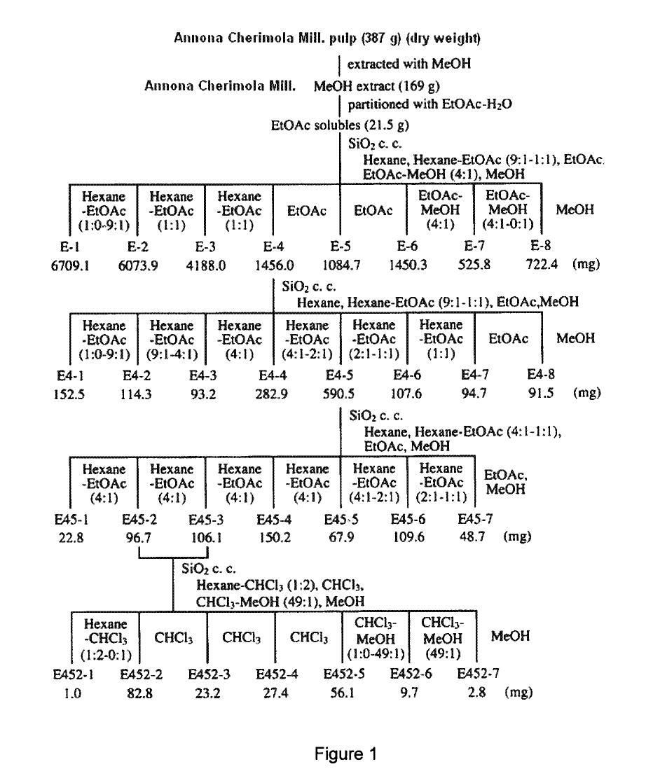

White solid substance AC-1 was isolated in the procedures shown in FIG. 1 from the ethyl acetate soluble fraction obtained in (i). First, the EtOAc soluble fraction (21.5 g) was applied to silica gel column chromatography, followed by sequential dissolution with hexane, hexane—EtOAc, EtOAc, EtOAc-MeOH, MeOH to yield fractions E-1 (6709.1 mg, hexane-EtOAc (1:0-9:1)). E-2 (6073.9 mg, hexane-EtOAc (1:1)). E-3 (4188.0 mg, hexane-EtOAc (1:1)). E-4 (1456.0 mg, EtOAc). E-5 (1084.7 mg, EtOAc), E-6 (1450.3 mg, EtOAc-MeOH(4:1)), E-7 (525.8 mg, EtOAc-MeOH(4:1-0:1)). E-8 (722.4 mg, MeOH). The spontaneous immunoactivity of the respective fractions was measured at sample concentration of 3.3 μg/mL in the procedure discussed in Example 1. The results indicated that fraction E-4 provided the strongest inhibition of spontaneous immunity.

Next, the E-4 fraction was applied to silica gel column chromatography and sequentially dissolved in hexane, hexane-EtOAc, EtOAc, MeOH to yield fractions E4-1 (152.2 mg, hexane-EtOAc (1:0-9:1)), E4-2 (114.3 mg, hexane-EtOAc (9:1-4:1)), E4-3 (93.2 mg, hexane-EtOAc (4:1)), E4-4 (282.9 mg, hexane-EtOAc (4:1-2:1)), E4-5 (590.5 mg, hexane-EtOAc (2:1-1:1)), E4-6 (107.6 mg, hexane-EtOAc (1:1)). E4-7 (94.7 mg, EtOAc), E4-8 (91.5 mg, MeOH). The spontaneous immunoactivity of the respective fractions was measured at sample concentration of 0.5 μg/mL, the results of which indicated that fraction E4-5 had the strongest inhibition of spontaneous immunity.

Next, fraction E4-5 was applied to silica gel column chromatography followed by sequential dissolution with hexane-EtOAc, EtOAc, MeOH to yield fraction E45-1 (22.8 mg, hexane-EtOAc (4:1)), E45-2 (96.7 mg, hexane-EtOAc (4:1)), E45-3 (106.1 mg, hexane-EtOAc (4:1)), E45-4 (150.2 mg, hexane-EtOAc (4:1)), E45-5 (67.9 mg, hexane-EtOAc (4:1-2:1)), E45-6 (109.6 mg, hexane-EtOAc (2:1-1:1)), E45-7 (48.7 mg, EtOAc, MeOH). The results of measuring the spontaneous immunoactivity of the respective fractions at sample concentration of 0.5 μg/mL revealed that fractions E45-2 and 3 had the strongest inhibition of spontaneous immunity.

Next, fraction E45-2 and E45-3 were mixed and applied to silica gel column chromatography followed by sequential dissolution with hexane-chloroform (CHCl₃), CHCl₃, CHCl₃-MeOH, MeOH to yield fractions E452-1 (1.0 mg, hexane-CHCl₃ (1:2-0:1)), E452-2 (82.8 mg, CHCl₃), E452-3 (23.2 mg, CHCl₃), E452-4 (27.4 mg, CHCl₃), E452-5 (56.1 mg, CHCl₃-MeOH (1:0-49:1)), E452-6 (9.7 mg, CHCl₃-MeOH (49:1)), E452-7 (2.8 mg, MeOH). The results of measuring the spontaneous immunoactivity of the respective fractions at sample concentration of 0.5 μg/mL revealed that fractions E452-3 and 4 had the strongest inhibition of spontaneous immunity.

Fractions E452-3 and 4, especially 452-3, had no effect on the cell survival rate at sample concentration of 5.0 μg/mL. The spontaneous immunoactivity of fractions E452-1 to 7 as well as the cell survival rates are presented below in Table 1.

TABLE 1

| | Fraction No. | | | | | | |
|---|---|---|---|---|---|---|---|
| | E452-1 | E452-2 | E452-3 | E452-4 | E452-5 | E452-6 | E452-7 |
| Spontaneous immunoactivity (%) | — | 61.9 | 39.3 | 18.8 | 97.7 | 108.0 | 123.9 |
| [Sample conc. 0.5 μg/mL] | | | | | | | |
| Cell survival rate (%) | — | 100.1 | 103.4 | 93.4 | 90.6 | 98.6 | 98.1 |
| [Sample conc. 5.0 μg/mL] | | | | | | | |

The results of analyzing fractions E452-3 and 4 using thin-layer chromatography revealed that two types of substances are found in these two fractions. Thus, fraction E452-3 was separated via HPLC (Column: JAIGEL-GS310, Elutant:CH₃CN, Flowrate: 5 mL/min) to yield fractions E4523-1 (0.3 mg), E4523-2 (18.8 mg), E4523-3 (0.5 mg), E4523-4 (1.1 mg). Next, fraction E4523-2 was separated via HPLC (Column: JAIGEL-1H, Elutant: CHCl₃, Flowrate:3.5 mL/min) to yield fractions E45232-1 (0.1 mg), E45232-2 (0.7 mg), E45232-3 (4.0 mg), E45232-4 (9.8 mg), E45232-5 (2.1 mg), E45232-6 (0.1 mg), E45232-7 (0.2 mg). Next, fractions E45232-3, 4, and 5 were mixed, applied to silica gel column chromatography and sequentially dissolved with hexane-EtOAc (1:0-4:1), EtOAc, MeOH to complete isolation of AC-1 (4.3 mg), a white solid, from the hexane-EtOAc (20:3) elution fraction.

(iii) Identification of Diterpene Compound 1

A molecular ion peak was found at m/z 392.2550 upon measurement of HREIMS for AC-1. This indicated that the molecular formula of AC1 is C₂₃H₃₆O₅.

Next, ¹H-NMR, ¹³C-NMR and HMQC spectra indicated the presence in AC-1 of two ester carbonyl carbons, one quaternary carbon to which one oxygen atom is bound, one oxymethylene carbon, one methoxyl carbon, three quaternary carbons, nine methylene carbons, three methine carbons and three methyl carbons.

Next, ¹H—¹H COSY indicated that AC-1 has the following partial structure:

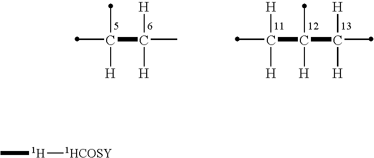

Thus, the A ring was initially analyzed by HMBC spectra.

The correlation from H-18 with C-3, -4, -5, and -19 was observed in the HMBC spectrum, and the results clarified the presence of bonds among C-3 to 5, 18, and 19. In addition, the correlation from protons of a methoxyl group to C-19 was observed, and the results indicated that C-19 forms a methoxycarbonyl. Furthermore, the correlation from H-20 to C-1, -5, -9 and -10 was observed, and the results clarified the presence of bonds among C-1, 5, 9, 10, 20. The correlation from H-1 to C-2, 3 was observed, and the results clarified the presence of bonds among C-1 to 3. AC-1 was demonstrated to have the following partial structure:

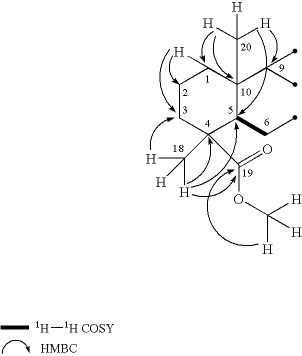

Next, the C ring section was analyzed next via HMBC spectra.

The correlation from H-17 to C-12, -15, -16, and a carbonyl carbon of an acetyl group was observed in the HMBC spectrum, and the results clarified the presence of bonds among C-12, 15, 16, 17 as well as an acetyl bonded to C-17. Furthermore, the correlation from H-13 to C-8, -14 as well as from H-15 to C-8 was observed, and the results clarified the presence of bonds among C-13-C-14-C-8-C-15. AC-1 was demonstrated to have the following partial structure:

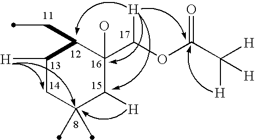

Finally, the correlation from H-11 to C-8 and C-9 as well as the correlation from H-15 to C-9 was observed in the HMBC spectrum, and the results clarified the presence of bonds among C-11-C-9-C-8. In addition, the correlation from H-6, 15 to C-7 as well as from H-7 to C-8 was observed, and the results clarified the presence of bonds among C-6-C-7-C-8. The aforementioned findings clarified the following planar structure:

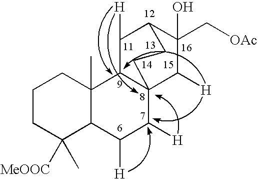

The compound in which a hydroxyl is bound at position 17 of AC-1 instead of an acetoxy (methyl 17-hydroxy-16β-hydroxy-ent-atisan-19-oate) is already known. Thus, potassium carbonate (K₂CO₃) was acted on AC-1 in methanol in order to compare said known compound with AC-1, and deactylation resulted in conversion of the acetoxy at position 17 of AC-1 into a hydroxyl to yield AC-1′. Measurement by ¹H-NMR, ¹³C-NMR and specific rotation ([α]D²⁴-63.9 (c 0.390, CHCl₃)) of AC-1′ derived in this manner revealed that the data matched those of the known compound ([α]D¹⁹-81.3 (c 0.8, CHCl₃)). Consequently, AC-1 could be confirmed to be diterpene compound 1 (methyl 17-acetoxy-16β-acetoxy-ent-atisan-19-oate).

¹H-NMR and ¹³C-NMR spectral data for AC-1 are presented in Table 2 below.

TABLE 2

| Positions | ¹³C(ppm) | ¹H(ppm) |
|---|---|---|
| 1 | 40.6 | 0.76(1H, dd, J=13.1, 4.2Hz) |
| | | 1.78(1H, m) |
| 2 | 19.0 | 1.40(1H, m) |
| | | 1.81(1H, m) |
| 3 | 38.0 | 0.96(1H, m) |
| | | 2.14(1H, d, J=13.5Hz) |
| 4 | 43.8 | |
| 5 | 56.8 | 1.00(1H, m) |
| 6 | 22.1 | 1.71(1H, dd, J=13.7, 3.0Hz) |
| | | 1.83(1H, m) |
| 7 | 41.8 | 1.42(1H, m) |
| | | 1.62(1H, m) |
| 8 | 44.8 | |
| 9 | 55.7 | 0.96(1H, m) |
| 10 | 39.4 | |
| 11 | 18.4 | 1.47(1H, m) |
| | | 1.57(1H, m) |
| 12 | 45.9 | 2.02(1H, s) |
| 13 | 37.1 | 1.63(1H, m) |
| | | 1.89(1H, m) |
| 14 | 26.2 | 1.47(2H, m) |
| 15 | 52.9 | 1.45(1H, m) |
| | | 1.54(1H, m) |
| 16 | 80.0 | |
| 17 | 68.5 | 4.21(2H, s) |
| 18 | 28.7 | 1.14(3H, s) |
| 19 | 178.0 | |
| 20 | 15.3 | 0.80(3H, s) |
| 17-OCOCH₃ | 20.9 | 2.08(3H, s) |
| 17-OCOCH₃ | 171.2 | |
| 19-OMe | 51.1 | 3.62(3H, s) |

^{a}600MHz for ¹H and 150MHz for ¹³C in CDCl₃.

### Example 4

### Production and Identification of Diterpene Compound 2 Represented by Formula (1)

### (i) Production of Diterpene Compound 2

Fraction E45-4 (150.2 mg) that exhibits strong spontaneous immunoactivity following fractions E45-2 and 3 was obtained similarly to the manner in Example 3. Fraction E45-4 had absolutely no effect on the cell survival rate at sample concentrations of 5.0 μg/mL. The spontaneous immunoactivity and cell survival rate of fractions R45-1 to 7 are presented in Table 3 below.

TABLE 3

| | Fraction No. | | | | | | |
|---|---|---|---|---|---|---|---|
| | E45-1 | E45-2 | E45-3 | E45-4 | E45-5 | E45-6 | E45-7 |
| Spontaneous immunoactivity (%) | 50.0 | 33.3 | 47.8 | 69.2 | 94.7 | 93.8 | 112.2 |
| [Sample conc. 0.5 μg/mL] | | | | | | | |
| Cell survival rate (%) | 102.1 | 102.7 | 95.4 | 97.7 | 97.9 | 99.4 | 103.1 |
| [Sample conc. 5.0 μg/mL] | | | | | | | |

Next, fraction E45-4 was applied to silica gel column chromatography followed by sequential dissolution with hexane-CHCl₃ (1:4), CHCl₃, CHCl₃-MeOH (9:1), MeOH to complete isolation of AC-2 (17.1 mg), a white solid, from the hexane-CHCl₃ (1:4) elution fraction.

### (ii) Identification of Diterpene Compound 2

Since a molecular ion peak was found at m/z 362.2440 upon measurement of HREIMS for AC-2, the molecular formula of AC-2 is surmised to be C₂₂H₃₄O₄.

Next, ¹H-NMR spectral measurement of AC-2 revealed that the signal (δ 3.62) of the methoxyl observed in the ¹H-NMR spectrum of AC-1 had vanished, and that a signal (δ 9.74) derived from aldehyde had appeared instead. Furthermore, measurement of the ¹³C-NMR of AC-2 indicated that the signals (δ 178.0 and 51.1) of a methoxycarbonyl group observed in the ¹³C-NMR spectrum of AC-1 had vanished, and that a signal (δ 205.7) of aldehyde had appeared instead.

The aforementioned results indicate that AC-2 has the following planar structure in which the methoxycarbonyl of AC-1 had been converted into an aldehyde:

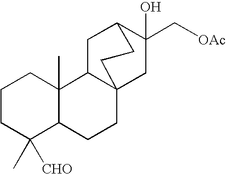

The compound in which a hydroxyl is bound instead of an acetoxy at position 17 of AC-2 (17-hydroxy-16β-hydroxy-ent-atisan-19-al) is already known. Thus, potassium carbonate (K₂CO₃) was acted on AC-2 in methanol in order to compare said known compound with AC-2, and deactylation resulted in conversion of the acetoxy at position 17 into a hydroxyl to yield AC-2′. Measurement by ¹H-NMR, ¹³C-NMR and specific rotation ([α]D²⁵-44.3 (c0.309, CHCl₃)) of AC-2 derived in this manner revealed that the data matched those of the known compound ([α]D²⁵-46.7 (c 0.15, CHCl₃)). Consequently, AC-2 could be confirmed to be diterpene compound 2 (17-acetoxy-16β-hydroxy-ent-atisan-19-al).

¹H-NMR and ¹³C-NMR spectral data of AC-2 are presented in Table 4 below.

TABLE 4

| Positions | ¹³C(ppm) | ¹H(ppm) |
|---|---|---|
| 1 | 39.7 | 0.78(1H, td, J=13.3, 4.2Hz) |
| | | 1.80(1H, m) |
| 2 | 18.3 | 1.44(1H, m) |
| | | 1.62(1H, m) |
| 3 | 34.2 | 0.97(1H, m) |
| | | 2.13(1H, m) |
| 4 | 48.4 | |
| 5 | 56.5 | 1.15(1H, dd, J=12.8, 2.1Hz) |
| 6 | 20.1 | 1.70(1H, m) |
| | | 1.90(1H, m) |
| 7 | 41.7 | 1.51(1H, m) |
| | | 1.71(1H, m) |
| 8 | 44.6 | |
| 9 | 55.1 | 1.02(1H, m) |
| 10 | 39.4 | |
| 11 | 18.3 | 1.50(1H, m) |
| | | 1.58(1H, m) |
| 12 | 45.9 | 2.05(1H, m) |
| 13 | 37.2 | 1.66(1H, m) |
| | | 1.91(1H, m) |
| 14 | 26.0 | 1.50(2H, m) |
| 15 | 52.8 | 1.49(1H, m) |
| | | 1.59(1H, m) |
| 16 | 79.9 | |
| 17 | 68.4 | 4.23(2H, s) |
| 18 | 24.2 | 0.99(3H, s) |
| 19 | 205.8 | 9.74(1H, d, J=1.4Hz) |
| 20 | 16.4 | 0.88(3H, s) |
| 17-OCOCH₃ | 20.9 | 2.11(3H, s) |
| 17-OCOCH₃ | 171.2 | |

^{a}600MHz for ¹H and 150MHz for ¹³C in CDCl₃.

### Example 5

### Production and Identification of Diterpene Compound 3 Represented by Formula (1)

The aldehyde bound at position 4 was converted into a hyroxymethyl by reducing diterpene compound 2 in MeOH using sodium borohydride (NaBH₄) to yield AC-3. The procedures that were carried out are presented below.

### (i) Production of Diterpene Compound 3

Fraction E45-2 (10.9 mg) that was obtained similarly to the method in Example 3 was dissolved in 2.0 mL of MeOH, followed by the addition of 1.7 mg of NaBH₄ and stirring for 20 minutes at room temperature. The reaction solution was acidified with saturated NH₄Cl solution, followed by extraction three times with EtOAc and solvent removal. The residue was applied to column chromatography loaded with 0.5 g of silica gel followed by sequential dissolution with CHCl₃, CHCl₃-MeOH (49:1), MeOH to yield AC-3 (4.2 mg) from the CHCl₃-MeOH (1:0-49:1) elution fraction.

### (ii) Identification of Diterpene Compound 3

The results of analysis using specific rotation, ¹H-NMR, ¹³C-NMR, LREIMS and HREIMS confirmed that AC-3 is diterpene compound 3 (17-acetoxy-ent-atisan-16β, 19-diol).

Specific rotation, ¹H-NMR, ¹³C-NMR, LREIMS and HREIMS data on AC-3 are presented below.

Colorless amorphous solid. [α]D²⁷-50.9 (c 0.305, pyridine), ¹H-NMR (600 MHz, pyridine-d₅) δ 5.75-5.87 (1H, br. s), 5.53-5.67 (1H, br. s), 4.65 (1H, d, J=11.2 Hz), 4.48 (1H, d, J=11.2 Hz), 3.99 (1H, d, J=10.6 Hz), 3.63 (1H, d, J=10.6 Hz), 2.40 (1H, s), 2.17 (1H, d, J=13.1 Hz), 2.00 (3H, s), 1.97 (1H, d, J=3.5 Hz), 1.94 (1H, d, J=11.8 Hz), 1.82 (1H, d, J=14.0 Hz), 1.72 (1H, d, J=14.0 Hz), 1.37-1.70 (11H, m), 1.19 (3H, s), 0.92-1.02 (3H, m), 1.00 (3H, s), 0.76 (1H, td, J=13.1, 4.2 Hz), ¹³C-NMR (150 MHz, pyridine-d₅) δ 171.2, 79.2, 69.3, 64.1, 57.2, 56.9, 54.0, 46.5, 45.0, 42.9, 40.6, 39.6, 39.3, 37.6, 36.2, 28.1, 26.7, 21.1, 20.9, 18.7, 18.6, 18.5. LREIMS m/z 364 (0.03, M⁺), 346 (3), 315 (32), 291 (43), 273 (82), 255 (100), HREIMS m/z 364.2614 [M]⁺ (364.2622 calculated for C₂₂H₃₆O₄).

### Example 6

### Production and Identification of Diterpene Compounds 4 to 6 Represented by Formula (1)

The acetoxy bound at position 17 was converted into a hydroxyl by deacetylation through the action of potassium carbonate (K₂CO₃) on diterpene compounds 1 to 3 in MeOH to yield diterpene compounds 4 to 6. The procedures that were carried out are presented below.

### (i) Production of Diterpene Compound 4

Diterpene compound 1 (6.2 mg) that was produced in Example 3 was dissolved in 2.0 mL of MeOH, followed by the addition of K₂CO₃ (2.6 mg) and stirring for 2 hours at room temperature. The MeOH was removed, followed by the addition of EtOAc and water to the residue. The solvent of the EtOAc layer was then removed. The residue was applied to column chromatography loaded with 0.5 g of silica gel followed by sequential dissolution with CHCl₃, CHCl₃-MeOH (99:1), MeOH to yield AC-4 (4.2 mg) from the CHCl₃-MeOH (99:1) elution fraction.

### (ii) Production of Diterpene Compound 5

Fraction E45-2 (8.7 mg) that was obtained similarly to the method in Example 3 was dissolved in 2.0 mL of MeOH, followed by the addition of 2.5 mg of K₂CO₃ and stirring for 2 hours at room temperature. The MeOH was removed, followed by the addition of EtOAc and water to the residue. The solvent of the EtOAc layer was then removed. The residue was applied to column chromatography loaded with 0.5 g of ODS followed by sequential dissolution with H₂O—CH₃CN (2:1-1:2) and MeOH to yield H₂O—CH₃CN (2:1). This fraction was then applied to column chromatography loaded with 0.5 g of silica gel, followed by sequential dissolution with CHCl₃, CHCl₃-MeOH (99:1-9:1), and MeOH to yield 1.2 mg of AC-5 from the CHCl₃-MeOH (99:1) elution fraction.

(iii) Production of Diterpene Compound 6

Fraction E45-2 (9.8 mg) that was obtained similarly to the method in Example 3 was dissolved in 2.0 mL of MeOH, followed by the addition of 1.8 mg of NaBH₄ and stirring for 20 minutes at room temperature. The solvent was removed followed by repeated dissolution in 2.0 mL of MeOH, and addition of 3.1 mg of K₂CO₃ and stirring for 1 hour at room temperature. The MeOH was removed, followed by the addition of EtOAc and water to the residue. The solvent of the EtOAc layer was then removed. The residue was applied to column chromatography loaded with 0.5 g of silica gel followed by sequential dissolution with hexane, hexane-EtOAc (4:1-1:1), EtOAc and MeOH to yield hexane-EtOAc (1:1) to yield 2.5 mg of AC-6 from the EtOAc, MeOH elution fraction.

### (iv) Identification of Diterpene Compounds 4-6

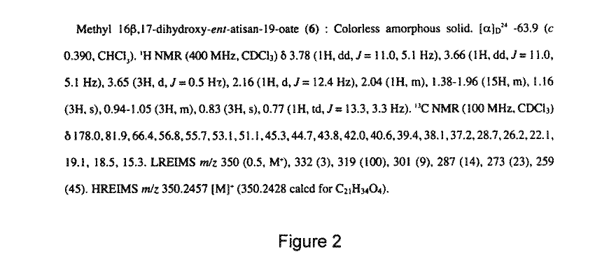

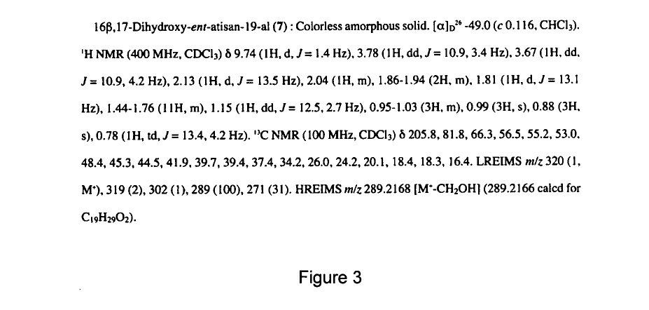

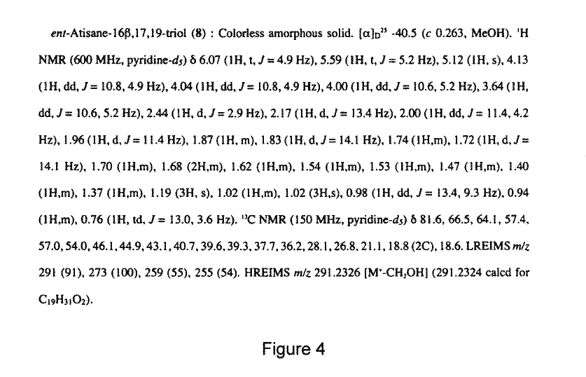

The results of analysis using specific rotation, ¹H-NMR, ¹³C-NMR, LREIMS and HREIMS confirmed that AC-4-6 are diterpene compounds 4-6, respectively. FIGS. 2 to 4 present the specific rotation, ¹H-NMR, ¹³C-NMR, LREIMS and HREIMS data.

### Example 7

### Confirmation 1 of the Cytokine Production Control Effect of Diterpene Compounds 1 to 6

### (i) Trial Method

The following trials were carried out to confirm the production control effect on IL-8 by diterpene compounds 1 to 6.

Eighteen samples were prepared by adding diterpene compounds 1 to 6 to HUVEC incubated in 96 well plates so as to reach 1.5 μM, 15 μM, and 150 μM, followed by incubation for 3 hours. That was followed by the addition of TNF-α (1 ng/ml) and incubation for 16 hours, after which the IL-8 concentration in the culture solution was measured using an ELISA kit (R&D systems).

Samples without human TNF-α or diterpene compounds added as well as samples to which only human TNF-α had been added without addition of diterpene compound were prepared for comparison.

### (ii) Results

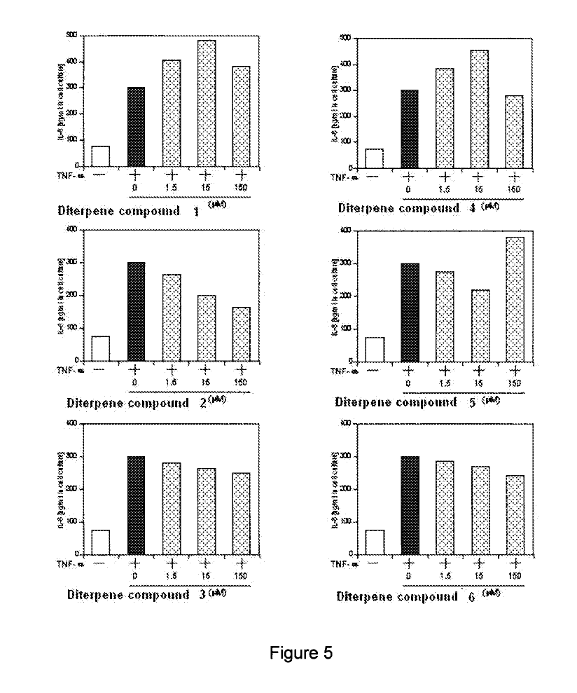

FIG. 5 presents the amount of IL-8 production in each sample. The ordinate in FIG. 5 shows the IL-8 concentration in culture solution while the abscissa shows the added concentration of each diterpene compound.

FIG. 5 confirms that diterpene compounds 1, 4 activate IL-8 production at low ranges of added concentrations while diterpene compounds 2, 3, 5 and 6 inhibit IL-8 production.

In addition, diterpene compound 5 switched its IL-8 production control effect from inhibition to activation at high ranges of added concentrations.

### Example 8

### Confirmation 2 of the Cytokine Production Control Effect of Diterpene Compounds 1 to 6

### (i) Trial Method

The following trials were carried out to confirm the production control effect on MCP-1 by diterpene compounds 1 to 6.

Eighteen samples were prepared by adding diterpene compounds 1 to 6 to HUVEC incubated in 96 well plates so as to reach 1.5 μM, 15 μM, and 150 μM, followed by incubation for 3 hours. That was followed by the addition of TNF-α (1 ng/ml) and incubation for 16 hours, after which the MCP-1 concentration in the culture solution was measured using an ELISA kit (R&D systems). Samples without human TNF-α or diterpene compounds added as well as samples to which only human TNF-α had been added without addition of diterpene compound were prepared for comparison.

### (ii) Results

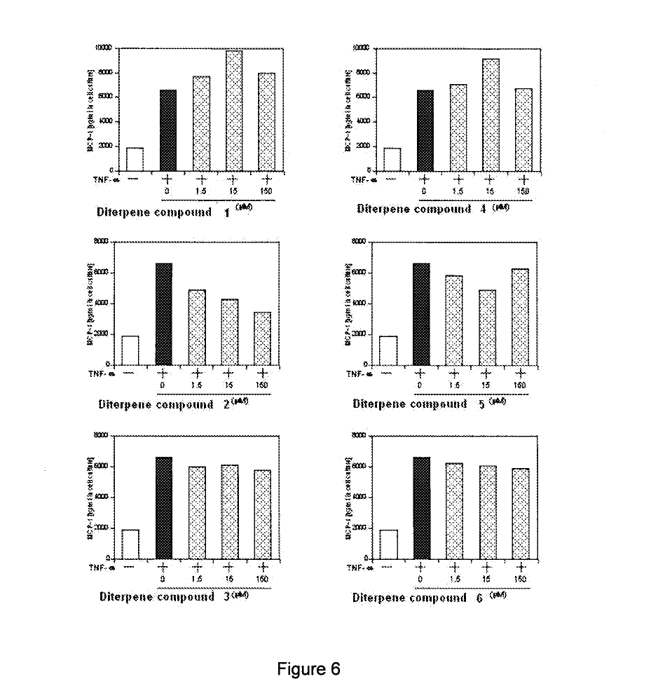

FIG. 6 presents the amount of MCP-1 production in each sample. The ordinate in FIG. 6 shows the MCP-1 concentration in culture solution while the abscissa shows the added concentration of each diterpene compound.

FIG. 6 confirms that diterpene compounds 1, 4 activate MCP-1 production at low ranges of added concentrations while diterpene compounds 2, 3, 5 and 6 inhibit MCP-1 production.

In addition, diterpene compound 5 lost its MCP-1 production control effect at high ranges of added concentrations.

Furthermore, comparisons with the results of Examples 7 and 8 revealed that diterpene compounds 1 to 6 exhibit production control effects of virtually identical tendencies on IL-8 and on MCP-1.

### Example 9

### Spontaneous Immunoactivity and Cytotoxicity of Diterpene Compounds 1 to 6 at Various Concentrations

For reference, the cytotoxicity and spontaneous immunoactivity of diterpene compounds 1 to 6 were measured at various concentrations. The spontaneous immunoactivity and cytotoxicity were measured by the same method as that stated in Example 1 and Example 2.

In addition, the transcription-translation activity (production level of β-galactosidase induced through thermal stimulation) of diterpene compounds 1 and 2 were measured. The transcription-translation activity was measured similarly to the method in Example 1 except in the following cases: when LPS was not added to the culture medium in “(i) Culture medium preparation”, when DMSO alone was added to the culture medium as a reference sample in “(vi) Evaluation”, when T-2 toxin was added to the culture medium as a blank sample so as to reach 100 μM, and when the following modifications of “(ii) Necropsy and incubation” were carried out.

Tubes holding Drosophilia larvae were cast into 30° C. incubators for 22 minutes in order to thermally stimulate the larvae. The larvae were decapitated at low-temperature conditions of not more than 4° C. to prevent further thermal stimulation, and the fat bodies, which are the organs that produce antibacterial peptide, were exposed. The resulting fat bodies were added, one each, to individual wells of a 96-well plate containing 100 μL of culture medium per well, followed by incubation for 18 hours at 25° C. Six Drosophilia were used per sample in incubation.

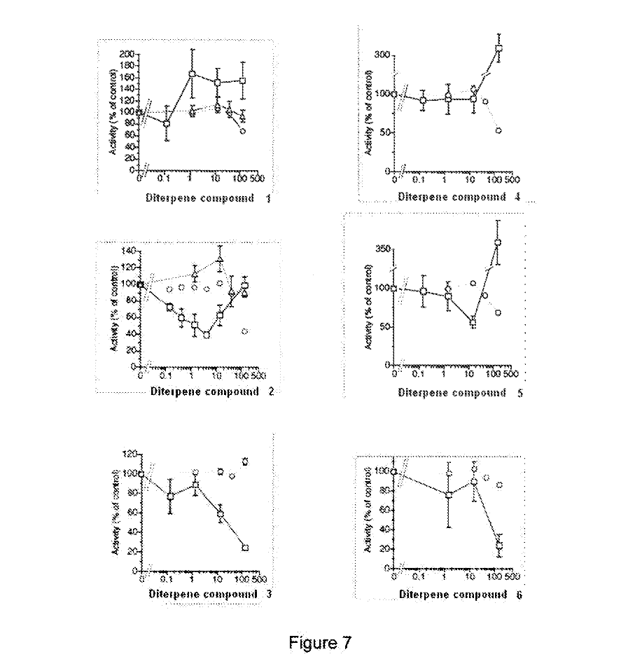

FIG. 7 shows the results. The symbols □, ∘, and Δ in FIG. 7 represent the spontaneous immunoactivity, cell survival rate, and transcription-translation activity, respectively. The ordinate shows the spontaneous immunoactivity, cell survival rate, or transcription-translation activity of each diterpene compound (relative figure versus reference sample (%)) while the abscissa shows the added concentration of each diterpene compound.

Patent application pursuant to the fruits of consigned research by the state and other entities (pursuant to Law on Special Measures for Industrial Revitalization, Article 30, National Agriculture and Food Research Organization “Agency for Promotion of Basic Research on New Technology—Exploitation of New Fields”).

What is claimed is:

1. A pharmaceutical composition comprising at least one diterpene compound of formula (1) 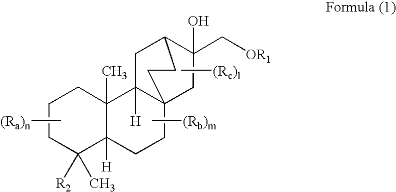
wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

2. The pharmaceutical composition of claim 1 wherein R₁ is selected from hydrogen and acetyl.

3. The pharmaceutical composition of claim 1 or 2 wherein R₂ is selected from methoxycarbonyl, aldehyde, and hydroxymethyl.

4. The pharmaceutical composition of claim 1 wherein the diterpene compound of formula (1) is a diterpene compound of formula (2)

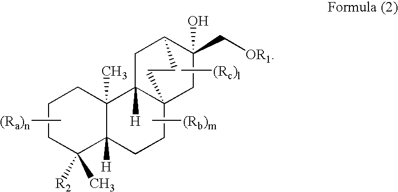

5. A method for treating or preventing a disease attributable to abnormal production of at least one cytokine comprising administering to a patient in need thereof a therapeutically effective amount of at least one pharmaceutical composition of any one of claims 1 to 4.

6. The method of claim 5 wherein the disease is chosen from viral infections, myocardial infarction, rheumatism, osteoporosis, arteriosclerosis, diabetes complications, sepsis, multiple myeloma, cervical cancer, post-organ transplant rejection response, hepatocirrhosis, acquired immune deficiency syndrome (AIDS), and multiple sclerosis.

7. The method of claim 5 wherein the disease is characterized by compromised immunity.

8. The method of claim 7 wherein the disease characterized by compromised immunity is selected from influenza and Spanish flu.

9. A composition for controlling cytokine production comprising at least one diterpene compound of formula (1) 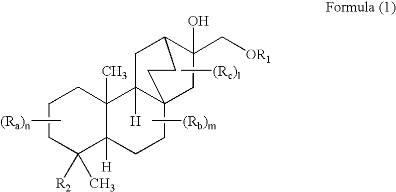
wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

10. The composition for controlling cytokine production of claim 9 wherein R₁ is selected from hydrogen and acetyl.

11. The composition for controlling cytokine production of claim 9 or 10 wherein R₂ is selected from methoxycarbonyl, aldehyde, and hydroxymethyl.

12. The composition for controlling cytokine production of claim 9 wherein the diterpene compound of formula (1) is a diterpene compound of formula (2)

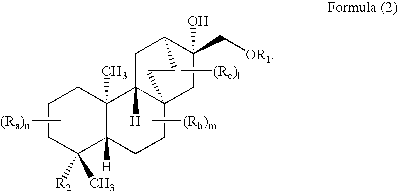

13. Food comprising a composition for controlling cytokine production of any one of claims 9 to 12.

14. A method for regulating cytokine production which comprises contacting said cytokine with an effective amount of at least one composition of any one of claims 9 to 12.

15. A method of producing one or more diterpene compounds of formula (1) comprising extracting parts of Annona Cherimola Mill., and isolating from the resulting extract one or more diterpene compounds of formula (1) 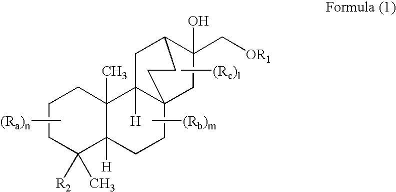
wherein
R₁ is selected from hydrogen, optionally substituted C₁ to C₆ acyl, and optionally substituted C₁ to C₆ hydrocarbon;
R₂ is selected from optionally substituted C₁ to C₆ alkoxycarbonyl, optionally substituted C₁ to C₆ acyl, and optionally substituted C₁ to C₆ hydrocarbon;
Rₐ, R_{b}, and R_{c} are independently optionally substituted C₁ to C₆ hydrocarbon;
n is an integer selected from 0, 1, 2, and 3;
m is an integer selected from 0, 1, and 2; and
l is an integer selected from 0, 1, 2, 3, 4, and 5.

16. The method of claim 15 further comprising converting one or more of the substituents of the isolated one or more diterpene compounds of formula (1) into other substituents giving one or more diterpene compounds of formula (1) with the desired cytokine production control action.

17. A diterpene compound of formula (3) 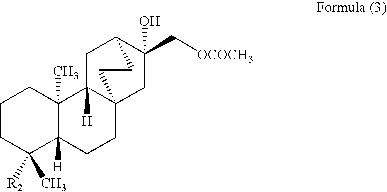
wherein
R₂ is selected from optionally substituted C₁ to C₆ alkoxycarbonyl, optionally substituted C₁ to C₆ acyl, and optionally substituted C₁ to C₆ hydrocarbon.

18. The diterpene compound of claim 17 wherein R₂ is selected from methoxycarbonyl, aldehyde, and hydroxymethyl.
